(19) United States
(12) Patent Application Publication
(10) Pub. No.: US 2003/0017550 A1
(43) Pub. Date: Jan. 23, 2003

(54) DNA sequences encoding fusion proteins comprising IFN-beta and TM-alpha1

(76) Inventor: Danny Zhong Der Pang, Fullerton, CA (US)

Correspondence Address: Danny Z. Pang, 1526 W. Flower Avenue, Fullerton, CA, 92833, US

(21) Appl. No.: 09/766,920

(22) Filed: Jan. 22, 2001

(51) Int. Cl. C12P 21/04; C07H 21/04; C12N 1/21; C12N 1/18; C12N 5/06; C12N 5/04; A61K 38/21

(52) U.S. Cl. 435/69.51; 435/320.1; 435/325; 435/348; 435/410; 435/252.3; 536/23.5; 435/254.2; 424/85.5

(57) ABSTRACT

Disclosed is a DNA sequence encoding fusion protein comprising human IFN-beta and human TM-alpha1. Such fusion proteins have the valuable biological activity of both constituents and the characteristics of being unique to viral, neoplastic, multiple sclerosis and immunodeficiency diseases. These proteins are thus useful for therapeutic purposes.

### BACKGROUND OF THE INVENTION

This invention relates generally to the field of DNA recombinant technology. More specifically, this invention relates to fusion proteins comprising human IFN-beta and human TM alpha1 and recombinant production of the fusion proteins and pharmaceutical compositions containing the fusion proteins.

Interferons (IFN) are a family of polypeptides synthesized and secreted by a large variety of eukaryotic cells in response to viral infections or to various synthetic and biological inducers, including viral constituents, double stranded RNA, and mitogens. Human IFNs are classified into two major groups. The IFNs-alpha, -beta, and -omega are designated type I IFNs, and IFN-gamma is designated type II IFN. Type I IFNs exhibit high homology in their primary, secondary, and tertiary structures. They interact with the same receptor and activate similar transcriptional signaling pathways, eliciting a similar range of biological responses, including antiviral, antiproliferative, and immunomodulatory activities. Binding to a quite distinct cell surface receptor than Type I IFN, Type-II IFN differs from type I-IFN in many aspects, such as the structure and induction of the gene, IFN's antigenicity, and biological responses. Type-II IFN has a different range of immune functions such as macrophage activation.

In the human IFN type-I group, there are at least 24 nonallelic genes or pseudogenes coding for structurally different forms of human IFN-alpha, while there is only a single gene coding for human IFN-beta which is situated on chromosome 9 in the same region as the IFN-alpha genes (Shows et al., Science 218:373, 1982; Trent et al., Proc. Natl. Acad. Sci. USA 79:7809, 1982; Owerback et al., Proc. Natl. Acad. Sci. USA 78:3123, 1981). Like the IFN-alpha, the IFN-beta gene does not contain intron. The gene encodes mature protein of 166 amino acids and contains an N-glycosylation site at position 180 (Derynck et al., Nature 285:542, 1980; Houghton et al., Nucleic Acids Res. 8:2885, 1980). Derived from the same ancestral gene, IFN-beta and IFN-alpha share a 30% amino acid or 45% nucleotide homology. Although IFN-beta and IFN-alpha bind to the same cell surface receptor, IFN-beta and IFN-alpha signal differently through their receptors since both INFs interact with the receptor subunits ifnar1 and ifnar2 in entirely different ways (Lewerenz et al., J. Mol. Biol. 282:585, 1998). Moreover, IFN-beta and IFN-alpha interact with the receptor in a distinct manner that leads to the differential biological responses (Russell-Harde et al., Biochem. Biophys. Res. Commun. 255:539, 1999). Therefore, despite type I interferon (IFN) subtypes alpha and beta share a common multicomponent cell surface receptor and elicit a similar range of biological responses, IFN-alpha and -beta exhibit key differences in several biological properties. For example, IFN-beta, but not IFN-alpha, induces the association of tyrosine-phosphorylated receptor components ifnar1 and ifnar2, and has activity in cells lacking the IFN receptor-associated, Janus kinase Tyk2 (Runkel et al., J. Biol. Chem. 273:8003, 1998). IFN-beta is also different from IN-alpha in induction, cellular origin, species specificity, as well as the physicochemical and serological properties.

Human IFN-beta (IFN-beta) is secreted by human primary fibroblasts after induction with virus, double-stranded RNA, or poly(I): poly(C) (Field et al., Biochemistry 58:1005, 1967; Weissenbach et al., Proc. Natl. Acad. Sci. USA 77:7152, 1980; Sehgal et al., Proc. Natl. Acad. Sci. USA 83:5219, 1986). IFN-beta is a glycoprotein of 166 amino acid residues with a molecular weight (MW) of 20,000-23,000 daltons (Knight E. Proc. Natl. Acad. Sci. USA 73:520, 1976; Tan et al., J. Biol. Chem. 254:8067, 1980; Knight and Diana J. Biol. Chem. 256:3609, 1981; Friesen et al., Arch. Biochem. Biophys. 206:432, 1981; Colby et al., J. Immunol. 133:3091, 1984; Novick et al., J. Gen. Virol. 64:905, 1983). The protein is encoded by a 0.9-kilobase (kb) mRNA which is derived from the intron-free IFN-beta gene on the short arm of chromosome 9 (Owerbach et al., Proc. Natl. Acad. Sci. USA 78:3123, 1981; Taniguchi et al., Gene 10:11,1980; Trent et al., Proc. Natl. Acad. Sci. USA 79:7809, 1982). The IFN-beta gene has been cloned and expressed in E. coli or mammalian cells as biologically active recombinant IFN-beta (Taniguchi et al., Nature 285:547, 1980; Itoh et al., DNA 3:157, 1980; McCullagh et al., J. Interferon Res. 3:97, 1983; McCormick et al., Mol. Cell. Biol. 4:166, 1984; Conradt et al., J. Biol. Chem. 262:1460, 1987; Scapol et al., J Chromatography 600:235, 1992). Nucleic acid and amino acid sequences of IFN-beta have also been determined (Houghton et al., Nucleic Acids Res. 8:2885,1980; Goeddel et al., Nucleic Acids Res. 8:4057, 1980; Sehgal, Biochim Biochys Acta 695:17, 1982).

IFN-beta is commercially available in recombinant forms, IFN-beta1a (Avonex, Biogen, Cambridge, Mass.) and IFN-beta1b (Betaseron, Berlex Laboratories, Richmond, Calif.). Whereas IFN-beta1a is a glycosylated molecule produced in a Chinese hamster ovarian (CHO) cell line containing the natural human IFN-beta amino acid sequence, IFN-beta1b produced in E. coli is a unglycosylated IFN-beta containing a genetically engineered serine substitution for cystine at position 17 (Mark et al., Proc. Natl. Acad. Sci. USA 81:5662, 1984). Ranging from 17,000 to 19,000 dalton, unglycosylated E. coli-derived IFN-beta has a smaller MW than glycosylated IFN-beta (Colby et al., J. Immunol. 133:3091,1984; Whitehorn et al., Gene 36:375, 1985; Gross et al., Biochi. Biophy. Acta 825:207, 1985; Remaut et al., Methods Enzymol. 119:366, 1986; Utsumi et al., J. Biochem. 101:1199, 1987). Replacing the cysteine at residue number 17 with a serine residue seems to be able to stablize the biologic activity of E.coli-derived IFN-beta (Mark et al., Proc. Natl. Acad. Sci. USA 81:5662, 1984). Despite their differences in the hydrophobic and the electrostatic properties, E. coli-derived IFN-beta and CHO-derived IFN-beta showed a similar higher-order structure and similar biological activities (Derynck et al., Nature 287:193, 1980; Utsumi et al., J. Biochem. 99:1533, 1986; Utsumi et al., J. Biochem. 101:1199, 1987). It has been reported that both recombinant IFNs-beta with equal numbers of units show almost identical antiproliferative and antiviral effects on human myelin basic protein-reactive T-cell lines (Weber et al., Neurology 52:1069, 1999).

IFN-beta exhibits various biological and immunological activities and has potential applications in antiviral, anticancer, and immunotherapies. IFN-beta is the first cytokines to be applied clinically in human malignant diseases such as osteosarcoma, cervical dysplasia, basal cell carcinoma, acute myeloid leukemia, glioma, multiple myeloma and Hodgkin's disease (Fine et al., Clin. Cancer Res. 3:381,1997; Nagao et al., Hum. Cell 10:95, 1997; Wadler et al., Cancer J. Sci. Am. 4:331,1998). IFN-beta can cause local tumor regression when injected into subcutaneous tumoral nodules in melanoma and breast carcinoma-affected patients.

IFN-beta is also the first drug licensed for the treatment of multiple sclerosis (MS), a chronic disease characterized pathogenically by an immunoinflammatory reaction that is driven against the central nervous system myelin by T lymphocytes and macrophages (Olsson et al., Immunol. Rev. 144:245, 1995). IFN-beta treatment for MS results in reducing exacerbation frequency, reducing progression of physical disability, reducing gadolinium-enhancing MRI brain lesions, and reducing accumulation of MRI T2 lesion volume. Although the pharmacokinetics of IFN-beta in MS patients are not fully understood, the therapeutic benefit of IFN-beta on MS might account for its' immunomodulatory and anti-inflammatory effects (Aranson, Neurology 43:641, 1993; Jiang et al., J. Immunol. 61:17, 1995; Yong et al, Neurology 51:682, 1998; Coclet-Ninin et al., Eur. Cytokine Netw. 8:345, 1997; Triantaphyllopoulos et al., Arthritis Rheum. 42:90, 1999; Weber et al., Neurology 52:1069, 1999; Rudick et al., Neurology 50:1294, 1998; Nicoletti et al., Clin. Exp. Immunol. 113:96, 1998; Ossege et al., J. Neuroimmunol. 91:73, 1998).

IFN-beta has been clinically tested in a variety of viral infections, such as genital warts and condylomata of the uterine cervix; viral encephalitis; herpes genitalis; herpes zoster; herpetic keratitis; herpes simplex; cytomegalovirus pneumonia, and viral hepatitis caused by hepatitis B virus (HBV) and hepatitis C virus (HCV) (Montalto et al., Am. J. Gastroenterol. 93:950, 1998; Musch et al., Hepatogastroenterology 45:2282, 1998). Recent results from clinical trials have indicated that IFN-beta administered by intravenous infusion can be sucessful in treating chronic HCV patients unresponsive to IFN-alpha therapy (Barbaro et al., Scand. J Gastroenterol. 34:928, 1999; Oketani et al., J. Clin. Gastroenterol. 28:49,1999; Vezzoli et al., Recenti. Prog Med. 89:235, 1998; Montalto et al., Am. J. Gastroenterol. 93:950, 1998). In the effect of early clearance of HCV RNA from the blood, IFN-beta seems even better than IFN-alpha in some cases (Furusyo et al., Dig. Dis. Sci. 44:608, 1999). IFN-beta seems also to be an effective retreatment therapy for children with chronic hepatitis B who are nonresponders to a first IFN-alpha cycle (Ruiz-Moreno et al., Pediatrics 99:222, 1997). Because of IFN-beta tolerance, higher doses and alternate routes of injection could be beneficial for the treatment of HBV and HCV.

A standard cell-free protein extract preparation from the thymus gland, known as thymosin fraction V (TF5) (U.S. Pat. No. 4,082,737), was demonstrated to be a potent immunopotentiating preparation. TF5 can suppress to various extents immune deficiency diseases and can also act in lieu of the thymus gland to reconstitute immune functions in thymic deprived and/or immunodeprived individuals (Wara et al., N. Engl. J Med. 292: 70, 1975). Analytical polyacrylamide gel electrophoresis and isoelectric focusing have demonstrated that TF5 consists of a number of polypeptides termed thymosin, with molecular weights ranging from 1,000 to 15,000.

The first of these peptides to be purified to homogeneity and sequenced from TF5 was called thymosin alpha 1 (TM-alpha1) (Goldstein et al., Proc. Natl. Acad. Sci. USA 74:725, 1977; U.S. Pat. No. 4,079,127). The chemical synthesis of TM-alpha1 by solution and solid phase synthesis techniques is described in U.S. Pat. Nos. 4,148,788 and 5,856,440. Identical to the native TM-alpha1 in the biological potent and amino acid sequence with lacking the N-terminal acetyl group, recombinant TM-alpha1 can be produced in E. coli by recombinant DNA cloning techniques (Wetzel et al., Biochemistry 19:6096, 1980). TM-alpha1 analogs and derivatives also have been produced, U.S. Pat. Nos. 4,116,951 and 5,512,656. TM-alpha1 is a 28 amino acid acidic peptide with a molecular weight of 3,108 and a pI in the range of 4.0-4.3. TM-alpha1 maintains many of the biologic effects of TF5 and has been found to be 10 to 1,000 times more active than TF5 in a number of bioassay systems designed to measure the maturation and function of T lymphocytes.

A another thymic peptide with 113 amino acid was named ProTM-alpha, because it was thought to be a precursor to TM-alpha1. ProTM-alpha includes thymosin-alpha1 as its 28 N-terminal amino acids and possess the same approximate quantitative and qualitative biological activity that has been ascribed to TM-alpha1 (U.S. Pat. No. 4,716,148; Smith, Leukemia and Lymphoma, 18:209, 1995).

TM-alpha1 potentiates the immune system by stimulating the production of IFN-alpha, IFN-gamma, macrophage migration inhibitory factor, interleukin-2 and interleukin-2 receptor, increasing T cell numbers and improving T-cell helper cell activity (Marshall, G. D., et al., J. Immunol. 126:741, 1981; Mutchnick, M. G., et al., Clin. Immunol. Immunopathol. 23:626, 1982; Low, T. L. K., et al., Thymus 6:27,1984; Sztein, M. B., et al., Proc. Nat'l Acad. Sci. U.S.A. 83:6107, 1986; Serrate, S. A., et al., J. Immunol. 1939:2338,1987; Baxevanis, C. N., et al., Immunopharm. 13:133, 1987; and, Svedersky, L. P., Eur. J Immunol. 12:244, 1982). TM-alpha1 is currently undergoing clinical trials in the U.S.A. as an immunomodulator in cancer patients, in individuals with chronic active hepatitis B, and as an immunoenhancer of vaccines in immunocompromised individuals. (Goldstein, A. L., Cancer Invest. 12:545, 1994; Lopez et al., Ann. Oncol. 5:741, 1994; Garaci et al., Eur. J Cancer. 31A:2403,1995; Garaci et al., Mech. Ageing. Dev. 96:103, 1997; Bonkovsky, H. L., Hepatology 26(3 Suppl 1):143S, 1997; Liaw, Y. F., J. Gastroenterol. Hepatol. 12:S346, 1997). TM-alpha1 has been approved for use in the treatment of hepatitis B in many Asian countries.

Experiments showed that TM-alpha1 specifically inhibits anchorage-independent growth of hepatitis B viral transfected HepG2 cells (Moshier et al., J. Hepatol. 25:814,1996). The clinical trials showed that TM-alpha1 is effective for the treatment of chronic hepatitis B and C, and more effective are the combination therapy of TM-alpha1 with IFN, due to the synergistic effects (Rost et al., Int. J Clin. Pharmacol. Ther. 37:51, 1999; Garaci et al., Int J Clin Lab Res, 24:23, 1994; Garaci et al., J Immunother, 13:7, 1993; Garaci et al., Eur. J. Cancer, 31A:2403, 1995; U.S. Pat. No. 5,849,696; Rasi et al., Gut, 39:679, 1996; Sherman et al., Hepatology, 27:1128, 1998; Moscarella et al., Liver 18:366, 1998). However, the efficacy of TM-alpha1, like most biologically active peptides, is hindered by its nature of a short half-life (Rost et al., Int. J Clin. Pharmacol. Ther. 37:51, 1999).

Many IFN-alpha hybrids, conjugates and chimeras are disclosed in an attempt to create IFN-alpha molecules with advantageous properties (U.S. Pat. Nos. 4,678,751; 5,071,761; 5,738,846; 5,594,107; Sperber et al., Antiviral. Res. 22:121, 1993; Rasch et al., Dig. Dis. Sci. 43:1719, 1998; He et al., J. Cancer Res. Clin. Oncol. 125:77, 1999). Hybrid proteins consisting of tumor necrosis factor-alpha and TM-alpha1 were reported to enhance the efficacy of vaccination against the causative agent of plague (Shmelev et al., Zh Mikrobiol Epideminol Immunobiol., 4:85, 1994). A biologically active single molecule of IFN-alpha and TM-alpha1 has been created by cross-linking chemically (Jeong and Chung, J. Biochem. Mol. Biol., 29:365, 1996). Recombinant fusion proteins comprising IFN-alpha2b and TM-alpha1 has also been indicated to retain the biological activities of both IFN-alpha2b and TM-alpha1 (U.S. patent application Ser. No. 09,333,348).

### BRIEF SUMMARY OF THE INVENTION

The present invention relates to a fusion protein comprising IFN-beta and TM-alpha1. The fusion proteins of this invention are represented by the following formulas:

I-T, T-I, I-L-T, or T-L-I

where I is IFN-beta; T is TM-alpha1; and L is a peptide linker. IFN-beta is fused to TM-alpha1 either directly or through a peptide linker. In preferred aspects, IFN-beta and TM-alpha1 are linked together via a linker to produce a single protein which retains the biological activity of IFN-beta and TM-alpha1. This invention also relates to pharmaceutical compositions containing the fusion molecules. The fusion proteins of the present invention may be characterized by possessing both biological properties of IFN-beta and TM-alpha1 or they may be further characterized by having a longer half-life and greater antiviral in vivo and stronger antiproliferative and immunomodulatory activities than their parental peptides. Such fusion proteins have the characteristics of being unique to viral, neoplastic and immunodeficiency diseases and are useful for therapeutic purposes.

### BRIEF DESCRIPTION OF THE SEVERAL VIEWS OF THE DRAWING

FIG. 1 shows the construction of plasmid pB/IFN-beta.

FIG. 2 shows the construction of plasmid pB/proTM-alpha1.

FIG. 3 is a schematic representation of the construction of plasmid pB/IFN-beta/TM-alpha1.

FIG. 4 is a schematic representation of the construction of plasmid pZDGU9.

FIG. 5 is a schematic representation of the construction of plasmid pZDGU10/IFN-beta/TM-alpha1.

### DETAILED DESCRIPTION OF THE INVENTION

The present invention is directed to fusion proteins comprising IFN-beta and TM-alpha1.

1. Definition

In describing the present invention, the following terms are intended to be defined as indicated below.

“Recombinant” polypeptides refer to polypeptides produced by recombinant DNA techniques; i.e., produced from cells (microbial or mammalian) transformed by an exogenous DNA construct encoding the desired polypeptide. Polypeptide expressed in most bacterial cultures. e.g., E. coli, will be free of glycan. Polypeptide expressed in yeast may have a glycosylation pattern different from that expressed in mammalian cells.

“Native” proteins or polypeptides refer to proteins or polypeptides recovered from a source occurring in nature. Thus, the term “native TM alpha1 ” would include naturally occurring TM alpha1 and fragments thereof.

A DNA “coding sequence” is a DNA sequence which is transcribed into mRNA and translated into a polypeptide in a host cell when placed under the control of appropriate regulatory sequences. The boundaries of the coding sequence are determined by a start codon at the 5′ N-terminus and a translation stop codon at the 3′ C-terminus. A coding sequence can include prokaryotic sequences, cDNA from eukaryotic mRNA, genomic DNA sequences from eukaryotic DNA, and even synthetic DNA sequences. A transcription termination sequence will usually be located 3′ to the coding sequence.

“Fusion protein” is a protein resulting from the expression of at least two operatively-linked heterologous coding sequences. The protein comprising IFN-beta peptide and TM-alpha1 peptide sequences of this invention is an example of a fusion protein.

“Nucleotide sequence” is a heteropolymer of deoxyribonucleotides (bases adenine, guanine, thymine, or cytosine). DNA sequences encoding the fusion proteins of this invention can be assembled from synthetic or cDNA-derived DNA fragments and short oligonucleotide linkers to provide a synthetic gene which is capable of being expressed in a recombinant expression vector. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

“Recombinant expression vector” is a replicable DNA construct used either to amplify or to express DNA encoding the fusion proteins of the present invention. An expression vector contains DNA control sequences and coding sequence. DNA control sequences include promoter sequences, ribosome binding sites, polyadenylation signals, transcription termination sequences, upstream regulatory domains and enhancers. Recombinant expression systems as defined herein will express the fusion proteins upon induction of the regulatory elements.

“Transformed host cells” refer to cells that have been transformed and transfected with exogenous DNA. Exogenous DNA may or may not be integrated (covalently linked) to chromosomal DNA making up the genome of the cell. In prokaryotes and yeast, for example, the exogenous DNA may be maintained on an episomal element, such as a plasmid or stably integrated into chromosomal DNA. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cell containing the exogenous DNA.

“PCR” means polymerase chain reaction which is based on a thermostable DNA polymerase from Thermus aquaticus. The PCR technique refers to a DNA amplification skill that mimics the natural DNA replication process in that the DNA molecules double after each thermal cycle, in a way similar to in vivo replication. The DNA polymerase mediates extension in a 5′ to 3′ direction. The “primer” refers to an oligonucleotide sequence that provides a 3′end to which the DNA polymerase adds nucleotides complementary to a nucleotide sequence. The “template” refers to a nucleotide sequence to which the primers are annealed.

2. Interferon Beta

The term interferon beta (IFN-beta) refers to proteins having amino acid sequences which are substantially similar to the native human IFN-beta amino acid sequences and which are biologically active in that they are capable of binding to IFN-beta receptors, transducing a biological signal initiated by binding IFN-beta receptors, or cross-reacting with anti-IFN-beta antibodies raised against IFN-beta. IFN-beta was selected as the fusion partner for the IFN-beta/TM-alpha fusion proteins of the invention, although any other IFN species can be used as well. IFN-beta polypeptides and DNA sequences encoding IFN beta are disclosed, for example, in Houghton et al., Nucleic Acids Res. 8:2885,1980 and Taniguchi et al., Gene 10:11,1980.

3. Thymosin alpha1

The term thymosin alpha 1 (TM-alpha1) refers to proteins having amino acid sequences which are substantially similar to the native human TM-alpha1 amino acid sequences and which are biologically active in that they are capable of binding to thymosin receptors, transducing a biological signal initiated by binding TM-alpha1 receptors, or cross-reacting with anti-TM-alpha1 antibodies raised against TM-alpha1. Such sequences are disclosed, for example, in U.S. Pat. No. 4,079,127.

The term “TM-alpha1” also includes analogs of TM-alpha1 molecules which exhibit at least some biological activity in common with native human TM-alpha1. Exemplary analogs of TM-alpha1 are disclosed in US. Patent Nos. 4,116,951; 4,466,918; and 5,512,656. Other TM-alpha1 analogs which are described herein may also be used to construct fusion proteins with thymosin. Furthermore, those skilled in the art of mutagenesis will appreciate that other analogs, as yet undisclosed or undiscovered, may be used to construct IFN-beta/TM-alpha1 fusion proteins as described herein.

4. Fusion Proteins Comprising IFN-beta and TM-alpha1

The term “fusion protein” herein refers to the proteins resulting from the expression of IFN-beta and TM-alpha1 operatively-linked heterologous coding sequences. The fusion proteins of the present invention include constructs in which the C-terminal portion of IFN-beta is fused to the N-terminal portion of TM-alpha1, and also constructs in which the C-terminal portion of TM-alpha1 is fused to the N-terminal portion of IFN-beta. IFN-beta is fused to TM-alpha1 either directly or through a linker. Specifically, the fusion proteins of the present invention are represented by the following formulas:

I-T, T-I, I-L-T, or T-L-I

where I is IFN-beta; T is TM-alpha1; and L is a peptide linker. Specific fusion protein constructs are named by listing the IFN-beta and TM-alpha1 domains in the fusion protein in their order of occurrence (with the N-terminal domain specified first, followed by the C-terminal domain). Thus, IFN-beta/TM-alpha1 refers to a fusion protein comprising IFN-beta followed by TM-alpha1 (i.e., the C-terminus of IFN-beta is fused to the N-terminus of TM-alpha1). Unless otherwise specified, the terms IFN-beta/TM-alpha1 and TM-alpha1/IFN-beta refer to fusion proteins with a peptide linker added. IFN-beta is fused to TM-alpha1 in such a manner as to produce a single protein which retains the biological activity of both IFN-beta and TM-alpha1.

Examples of fusion proteins comprising IFN-beta and TM-alpha1 are shown in the accompanying Sequence Listing. SEQ ID NO:10 shows the nucleotide sequence and corresponding amino acid sequence of a human IFN-beta/TM-alpha1 fusion protein. The fusion protein comprises human IFN-beta (amino acids 1-166) linked to human TM-alpha1 (amino acids 172-199) via a linker sequence (amino acids 167-171), as shown in SEQ ID NO:11.

Equivalent fusion proteins may vary from the sequence of SEQ ID NO:10 and SEQ ID NO: 11 by one or more substitutions, deletions, or additions, the net effect of which is to retain biological activity of the protein when derived as a fusion protein comprising IFN-beta and TM-alpha1.

5. Construction of cDNA Sequences Encoding Fusion Proteins Comprising IFN-beta and TM-alpha1

A DNA sequence encoding a fusion protein is constructed using recombinant DNA techniques to assemble separate DNA fragments encoding IFN-beta and TM-alpha1 into an appropriate expression vector. For example, the 3′ end of a DNA fragment encoding IFN-beta is ligated to the 5′ end of the DNA fragment encoding TM-alpha1, with the reading frames of the sequences in phase to permit mRNA translation of the sequences into a single biologically active fusion protein. The resulting protein is fusion protein comprising IFN-beta and TM-alpha1. Alternatively, the 3′ end of a DNA fragment encoding TM-alpha1 may be ligated to the 5′ end of the DNA fragment encoding IFN-beta, with the reading frames of the sequences in phase to permit mRNA translation of the sequences into a single biologically active fusion protein. The regulatory elements responsible for transcription of DNA into mRNA are retained on the first of the two DNA sequences, while stop codons, which would prevent read-through to the second DNA sequence, are eliminated. Conversely, regulatory elements are removed from the second DNA sequence while stop codons required to end translation are retained.

The IFN-beta is fused to TM-alpha1 with or without a linker. In preferred aspects of the present invention, the IFN-beta and TM-alpha1 constituents are linked through a peptide linker consisting of 1 to about 15 genetically encodable amino acids. Preferred peptide linker sequence comprises amino acid selected from the group consisting of Ser, Gly, Ala, Thr, Pro and Ile.

The linker sequence is incorporated into the fusion protein construct by well known standard PCR extension methods as described below.

6. Proteins and Analogs

The present invention provides a fusion protein comprising human IFN-beta and human TM-alpha1. Derivatives and analogs of the fusion proteins of the present invention may also be obtained by modifying the primary amino acid structure with other chemical moieties, by mutations of the fusion protein, by linking particular functional groups to amino acid side chains or at the N- or C-termini, or by conjugating the fusion protein with other proteins or polypeptides. Bioequivalent analogs of the fusion proteins may also be constructed by making various substitutions of residues or sequences.

7. Expression of Recombinant Fusion Proteins Comprising IFN-beta and TM-alpha1

There are several ways to express the recombinant fusion proteins in vitro, including in E. coli, baculovirus, yeast, mammalian cells or other expression systems.

The prokaryotic system, E. coli, is not able to do post-translational modification, such as glycosylation. But this is probably not a problem for the IFN-beta/TM-alpha1 fusion protein since the native IFN-beta and TM-alpha1 are not heavily glycosylated. Further, it has been reported that recombinant IFN-beta and TM-alpha1 without any glycosylation retained their biological activities (U.S. Pat. Nos. 4,737,462 and 5,814,485; Goeddel et al., Nucleic Acids Res. 8:4057, 1980; Wetzel et al., Biochemistry 19:6096, 1980; Utsumi et al., J. Biochem., 101:1199, 1987). With the prokaryotic system, the expressed protein is either present in the cell cytoplasm in an insoluble form so-called inclusion bodies or is found in the soluble fraction after the cell has been lysed (Thatcher & Panayotatos, Methods Enzymol., 119:166, 1986; Goeddel et al., Nature 287:411,1980; Dworkin-Rastl et al., Gene 21:237,1983). If the expressed protein is in insoluble inclusion bodies, solubilization and subsequent refolding of the inclusion bodies is usually required (Schein, C. H. and Notebom, H. M., Bio/technology, 6:291-294, 1988; Wilkinson, D. L. and Harrison, R. G., Bio/technology, 9:443-448, 1991).

Many prokaryotic expression vectors are known to those of the skill in the art, such as pKK223-3 (Pharmacia Fine Chemicals, Uppsala, Sweden), pKK233-2 (Clontech, Palo Alto, Calif., USA), and pGEM1 (Promega Biotech, Madison, Wis., USA), which are commercial available. Another exemplary prokaryotic expression vector is pZDGU, described in Example 2 below.

Promoters commonly used in recombinant microbial expression systems include the beta-lactamase (penicillinase) and lactose promoter system (Chang et al., Nature 275:6, 1978; Goeddel et al., Nature 281:544, 1979), the tryptophan (trp) promoter system (Goeddel et al., Nucl. Acids Res. 8:4057, 1980) and tac promoter (Maniatis, Molecular Cloning: A Laboratory Mannual, Cold Spring Harbor Laboratory, page 412, 1982). A particularly useful bacterial expression system employs the phage lamda P_{L} promoter and cIts857 thermoinducible repressor (Bernard et al., Gene 5:59, 1979; Remaut et al., Methods Enzymol. 119:366, 1986; Love et al., Gene 176:49, 1996), as described in Example 2 below.

Recombinant fusion proteins may also be expressed in yeast hosts such as Saccharomyces cerevisiae and Pichia pastoris. It usually gives the ability to do various post-translational modifications. The expressed fusion protein can be secreted into the culture supernatant where not many other proteins reside, making protein purification easier. Yeast vectors for expression of the fusion proteins in this invention contain certain requisite features. The elements of the vector are generally derived from yeast and bacteria to permit propagation of the plasmid in both. The bacterial elements include an origin of replication and selectable marker. The yeast elements include an origin of replication sequence (ARS), a selectable marker, promoter, and a transcription termination.

Suitable promoters in yeast vectors for expression include the promoters of the TRP1 gene, the ADHI or ADHII gene, acid phosphatase (PH03 or PH05) gene, isocytochrome gene, or the promoters involved with the glycolytic pathway, such as the promoter of enolase, glyceraldehyde-3-phosphate dehydrogenase (GAPDH), 3-phosphoglycerate kinase (PGK), hexokinase, pyruvate kinase, triosephosphate isomerase and phosphoglucose isomerase (Hitzeman et al., J. Biol. Chem. 255:2073, 1980; Hess et al., J Adv. Enzyme Reg. 7:149, 1968; and Holland et al., Biochem. 17:4900, 1978).

Commercial available yeast vectors include pYES2, pPIC9 (Invitrogen, San Diego, Calif.), YEpc-pADH2a, pYcDE-1 (Washington Research, Seattle, Wash.), pBC102-K22 (ATCC# 67255), and YpGX265GAL4 (ATCC# 67233).

Mammalian cell lines, such as the COS-7, L cells, C127, 3T3, Chinese hamster ovary (CHO), Hela and BHK, can be employed to express the recombinant fusion proteins in this invention. The recombinant proteins produced in mammalian cells are normally soluble and glycosylated and have an authentic N-terminal. Mammalian expression vectors may contain non-transcribed elements such as an origin of replication, promoter and enhancer, and 5′ or 3′ nontranslated sequences such as ribosome binding sites (RBS), a poly-adenylation site, acceptor sites and splice donor, and transcriptional termination sequences. Promoters for use in mammalian expression vectors usually are for example viral promoters, such as Polyoma, Adenovirus, HTLV, Simian Virus 40 (SV40), and human cytomegalovirus (CMV). An example of the mammalian expression vectors is pcDNA3, ((Invitrogen, San Diego, Calif.), which contains a CMV promoter and a NEO resistant gene.

Depending on the expression system and host selected, a homogeneous recombinant fusion protein can be obtained by some of the purification steps, in various combinations, of the conventional chromatographys of protein purification, which include affinity chromatography, reverse phase chromatography, cation exchange chromatography, anion exchange chromatography, hydrophobic interaction chromatography, gel filtration chromatography and high performance liquid chromatography (HPLC). If the expression system secretes the fusion protein into the growth media, the protein can be purified directly from the media. If the fusion protein is not secreted, it is isolated from cell lysates. Cell disruption can be done by any conventional method, including freeze-thaw cycling, sonication, mechanical disruption, or use of cell lysing agents.

Fusion protein compositions can be prepared for administration by combining fusion protein having the desired degree of purity and the pharmaceutically effective amount with physiologically acceptable carriers.

Fusion protein compositions may be used to enhance proliferation, maturation and functional activation of T cells, or to enhance antiviral, antiproliferative and immunomodulatory effects. Specifically, compositions containing the fusion protein may be used to enhance the immune system to battle against viral, neoplastic and immunodeficiency diseases. To achieve this result, a pharmaceutically effective quantity of a fusion protein composition is administered to a mammal, preferably a human, in association with a pharmaceutically acceptable carrier.

The following examples are offered to further illustrate the invention and are not intended to be limitative thereof:

### EXAMPLE 1

Synthesis of Expression Vectors Encoding an IFN-beta/TM-alpha1 Fusion Protein

1. Cell Culture and RNA Extraction

Peripheral blood monocytes (PBMs) were isolated from buffy coats by Ficoll-Hypaque density centrifugation. PBMs were repeatedly washed with sterile PBS (phosphate-buffered saline) and spinned down by centrifugation. The cells at 5 times.10.sup.6 cells/mil were cultured for 18 hours in 175 cm sup.2. flasks at 37.degree. C and 5% CO.sub.2 in air in 100 ml RPMI supplemented with 10% fetal calf serum, 1% phytohemagglutinin (PHA) and 100 units human rIL-2/ml. The cells suspended in the culture medium were transferred to a new flask with the original medium, then phorbol 12-myristate 13-acetate (PMA) was added to the culture at a final concentration of 50 .mu.g/ml. The adhesive cells as a monolayer on the botton surface of the flask were added with a fresh medium containing poly I:C 50 mu.g/mil and DEAE-destran 400 mu.g/ml. The cultures were continued for another 8 hours for the adhesive cell cultures at 37.degree. C and 5% CO.sub.2 in air before the cells were harvested by centrifugation. RNA was extracted by the guanidinium CsCl method and poly A⁺ RNA was prepared by oligo-dT cellulose chromatography (Maniatis et al., Molecular Cloning: A Laboratory Manual, Cold Spring Harbor, 1982).

The first-strand cDNA was then synthesized from poly A+RNA by reverse transcription (RT) using AMV reverse transcriptase with oligo(dT) as a 3′ primer in 10 mM Tris-HCl (pH 9.0), 50 mM KCl, 1.5 mM MgCl.sub.2 and 0.5 mM dNTPs in a total of 50 mu.l volume. The reaction mixture was incubated at a 42.degree. C. water bath for 60 minutes, followed by a dilution with 50.mu.l of DEPC treated water. After boiled for 3 minutes and cooled on ice for 2 minutes, the reaction mixture was used directly as the templates for PCR to amplify IFN-beta and ProTM-alpha cDNA, respectively.

2. Amplification and Cloning of cDNAs Encoding Human IFN-beta

The cDNA of IFN-beta was rendered double-stranded using Taq DNA polymerase and a set of upstream and downstream oligonucleotide primers for human IFN-beta. The primers used to amplify the INF-beta are shown in Table 1. The 5′ primer (IFN-beta-A) contained a NdeI site and the coding sequence for the first 8 amino acids from the IFN-beta. The 3′ primer (IFN-beta-B) contained a HindIII site and coding sequence for the last 6 amino acids from the IFN-beta. The PCR buffer contained 50 mM KCl, 10 mMTris-HCl (pH 9.0), 1.5 mM MgCl.sub.2, 0.01% gelatin, 0.05 mmol each of dNTP, 1.0 .mu.mol of each primers, 10.mu.l reverse transcription reaction mixture, and 2 units of Taq DNA polymerase in a total of 50.mu.l volume. The PCR condition was 94.degree. C. for 30 seconds, 55.degree. C. for 30 seconds, and 72.degree. C. for 30 seconds for 25 cycles in the MJ Research model PTC-1152 thermocycler (MJ Research, Watertown, Mass.).

TABLE 1
Primers used in PCR to amplify human IFN-beta

| Designation | Primer Sequence | | Primer Length |
|---|---|---|---|
| IFN-beta-A | 5′ ACATATGAGCTACACCTTGCTTGGATTC 3′ | (SEQ ID NO: 1) | 28 |
| IFN-beta-B | 5′ TAAGCTTTTTCACTTTCGGAGGTAACCTGT 3′ | (SEQ ID NO: 2) | 30 |

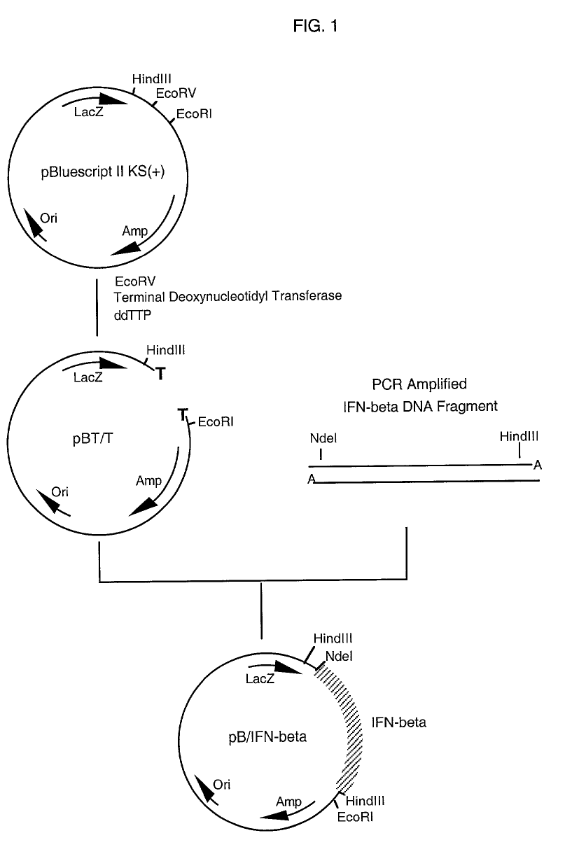

The PCR amplified DNA fragments were directly cloned into pBT/T vectors and then transformed into competent E.coli DH5.alpha. cells. pBT/T vector is derived from pBluescript II KS(+) cloning vector (Stratagene, La Jolla, Calif.). pBluescript II KS(+) was first cleaved by a restriction endonuclease EcoRV, then incubated with terminal deoxynucleotidyl transferase and ddTTP. The resulting vector pBT/T contains 3′-T overhangs at its MCS (multiple cloning sites) (FIG. 1). The PCR cDNA fragments with 3′-A overhangs can be ligated into pBT/T cloning vectors without any digestion of restriction endonuclease. Designing the restriction endonuclease sites NdeI and HindIII in the primers is for subcloning the cDNA fragments into expression vectors at the sites NdeI/HindIII.

The competent cells of DH5.alpha were prepared by the CaCl.sub.2 method (Mandel and Higa, J. Mol. Biol. 53:159, 1970). Briefly, 50 ml of LB medium without antibiotics is inoculated with a single E. coli DH5.alpha colony and grown overnight at 37.degree. C. with shaking at 250 rpm. The overnight culture is diluted 1:50 with LB medium without any antibiotic and continued the cultivation at 37.degree. C. with 250 rpm until an OD.sub.590 reaches 0.3-0.5. The culture is then placed on ice for 10 minutes and centrifuged 10 minutes at 3000 rpm at 4.degree. C. The supernatant is discarded. The cell pellet is resuspended gently in 40% of the starting volume with the ice-cold 0.1 M CaCl.sub.2 solution. The cell suspension is kept on ice for 30 minutes and then spinned down at 3000 rpm for 10 minutes at 4.degree. C. The pellet is resuspended again in 2% of the starting volume with the ice-cold 0.1 M CaCl.sub.2 solution, transferred into a sterile polypropylene tube, and then chilled on ice overnight at 4.degree. C. Cold sterile 80% glycerol in distilled water is added into the cell suspension to a final concentration of 20% and mixed gently. The competent cells, at a density of approximately 1 times10.sup.9/ml are stored in a 40 microliter aliquot at −70.degree. C.

Plasmide DNA was obtained from small overnight cultures by a modified alkaline lysis method (Lee and Rashid, BioTechniques 9:676, 1990). The size of the inserts was determined by digestion with restriction endonucleases NdeI and HindIII. DNA sequencing analysis in both directions with the primers shown in Table 2 by the chain termination method (Sanger et al., Pro. Natl. Acad. Sci. 74:5463, 1977) confirms that the DNA insert encodes a human IFN-beta. The plasmid containing the DNA insert encoding IFN-beta is designated as pB/IFN-beta (FIG. 1).

TABLE 2
Primers used for sequencing

| Designation | Primer Sequence | | Primer Length |
|---|---|---|---|
| T3 | 5′ ATTAACCCTCACTAAAG | (SEQ ID NO: 3) | 17 |
| T7 | 5′ TAATACGACTCACTATAGGG | (SEQ ID NO: 4) | 20 |

3. Amplification and Cloning of cDNAs Encoding Human proTM-alpha

The cDNA of the human proTM-alpha was also obtained by reverse transcription and PCR performed the same way as described above. The primers for the PCR are shown in Table 3. The 5′ primer (TM-A) contained a NcoI site and the coding sequence for the first 6 amino acids from TM-alpha1. The 3′ primer (proTM-B) contained a HindIII site and coding sequence for the last 6 amino acids from the proTM-alpha.

TABLE 3
Primers used in PCR to amplify human proTM-alpha

| Designation | Primer Sequence | | Primer Length |
|---|---|---|---|
| TM-A | 5′ AGCCATGGCATCAGACGCAGCCGTAGAC 3′ | (SEQ ID NO: 5) | 28 |
| proTM-B | 5′ CCAAGCTTTACTAGTCATCCTCGTCGGTCTT 3′ | (SEQ ID NO: 6) | 31 |

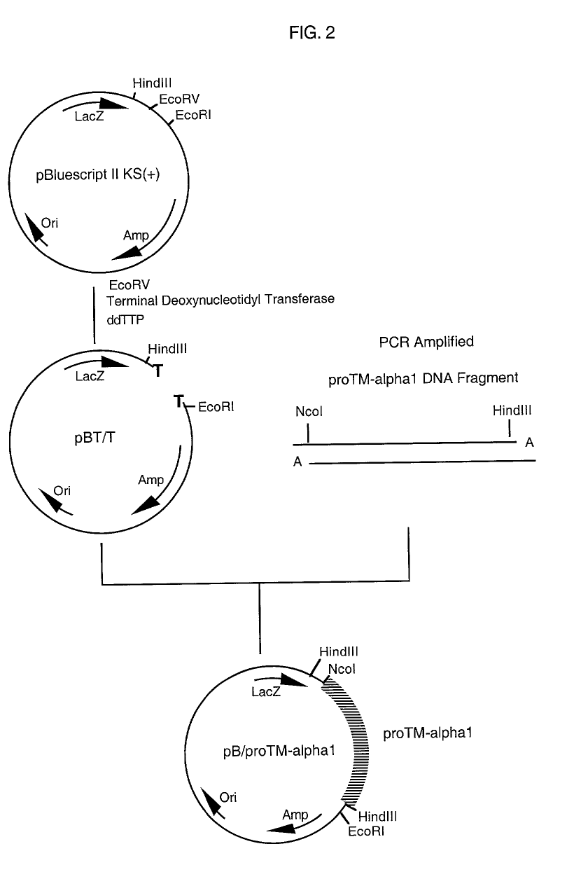

The PCR amplified DNA fragments were directly cloned into pBT/T vectors and then transformed into competent E.coli DH5.alpha. Isolation of plasmid DNA and determination of the size and the sequence of the DNA insert were performed as described above. The plasmid containing the DNA insert encoding proTM-alpha is designated as pB/proTM-alpha (FIG. 2).

4. Synthesis and Cloning of IFN-beta/TM-alpha1 Fusion cDNA

(a). Synthesis of cDNA Encoding IFN-beta and a Linker

pB/IFN-beta was prepared by the digestion with restriction endonuclease BamHI and used as a template for PCR to generate and amplify the cDNA containing the IFN-beta and a linker. The linker is attached to the 3′ end of IFN-beta and the fragment is named IFN-beta-L. PCR performed the same way as described above. The primers for PCR amplification are shown in Table 4. 5′ primer (IFN-beta-A) contained a NdeI site and the coding sequence for the first 8 amino acids from the IFN-beta. The 3′ primer (IFN-beta-L-B) contained the sequence coding for a linker and the last 6 amino acids from the IFN-beta.

TABLE 4
Primers used in PCR to generate and amplify IFN-beta-L

| Designation | Primer Sequence | | Primer Length |
|---|---|---|---|
| | IFN-beta | | |
| IFN-beta-A | 5′ ACATATGAGCTACACCTTGCTTGGATTC 3′ | (SEQ ID NO: 1) | 28 |
| | Linker ---(3′ end) IFN-beta--- | | |
| IFN-beta-L-B | 5′ AGAGCCACCGCCACCCGAGTTTCGGAGGTAACCTGT 3′ | (SEQ ID NO: 7) | 36 |

The amplified PCR products named IFN-beta-L were gel-purified and stored at −20.degree. C. until used for preparation of IFN-beta/TM-alpha1 cDNA.

(b). Synthesis of cDNA Encoding TM-alpha1 and a Linker

pB/proTM-alpha was prepared by digestion with restriction enzyme BamHI and used as a template for PCR to generate and amplify the cDNA containing TM-alpha1 and a linker. The linker is attached to the 5′end of TM-alpha1 and the fragment is named L-TM-alpha1. PCR performed the same way as described above. The primers for PCR amplification are shown in Table 5. 5′ primer (L-TM-A) contained the sequence coding for a linker and the first 7 amino acids from the TM-alpha1. The 3′ primer (TM-B) contained a HindIII site and the coding sequence coding for the last 6 amino acids from the TM-alpha1.

TABLE 5
Primers used in PCR to generate and amplify L-TM-alpha1

| Designation | Primer Sequence | | Primer Length |
|---|---|---|---|
| | Linker ---(5′ end) TM-alpha1--- | | |
| L-TM-A | 5′ TCGGGTGGCGGTGGCTCTGACGCAGCCGTAGACACC 3′ | (SEQ ID NO: 8) | 36 |
| | TM-alpha1 | | |
| TM-B | 5′ TAAGCTTTACTAATTTTCTGCCTCTTCCAC 3′ | (SEQ ID NO: 9) | 30 |

The amplified PCR products named L-TM-alpha1 were gel-purified and stored at −20.degree. C. until used for preparation of IFN-beta/TM-alpha1 cDNA.

(c). Synthesis of cDNA Encoding IFN-beta/TM-alpha1 and its Expression Construct

IFN-beta/TM-alpha1 fusion cDNA was generated by PCR using the mixture (1:1 ratio) of IFN-beta-L and L-TM-alpha1 as templates. PCR performed the same way as described above. The primers for PCR amplification are shown in Table 6. 5′ primer (IFN-beta-A) contained a NdeI site and the coding sequence for the first 8 amino acids from the IFN-beta. The 3′ primer (TM-B) contained a HindIII site and the coding sequence for the last 6 amino acids from the TM-alpha1.

TABLE 6
Primers used in PCR to generate and amplify fusion cDNA,
IFN-beta/TM-alpha1

| Designation | Primer Sequence | | Primer Length |
|---|---|---|---|
| | IFN-beta | | |
| IFN-beta-A | 5′ ACATATGAGCTACACCTTGCTTGGATTC 3′ | (SEQ ID NO: 1) | 28 |
| | /NdeI | | |
| | TM-alpha1 | | |
| TM-B | 5′ TAAGCTTTACTAATTTTCTGCCTCTTCCAC 3′ | (SEQ ID NO: 9) | 30 |
| | /HindIII | | |

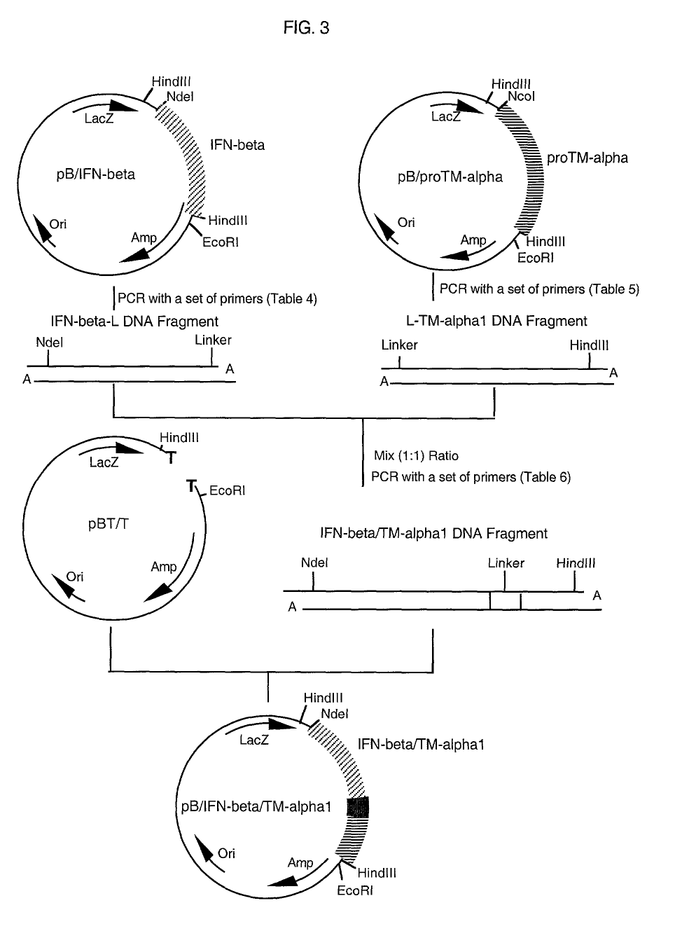

IFN-beta/TM-alpha1 DNA fragments were generated via PCR amplification and gel-purified. The IFN-beta/TM-alpha1 DNA was cloned into pBT/T vector and then transformed into competent E. coli strain DH5.alpha.cells. Isolation of the plasmid DNA and determination of the size and the sequence of the DNA insert were performed as described above. The DNA sequencing confirms that IFN-beta and TM-alpha1 is fused together via a 5 amino acids linker with the correct reading frames in phase. The nucleotide sequence and corresponding amino acid sequence of the fusion protein is shown in SEQ ID NO:10. The plasmid containing the DNA insert encoding IFN-beta/TM-alpha1 fusion protein is designated as pB/IFN-beta/TM-alpha1 (FIG. 3).

### EXAMPLE 2

Expression and Purification of IFN-beta/TM-alpha1 Fusion Protein

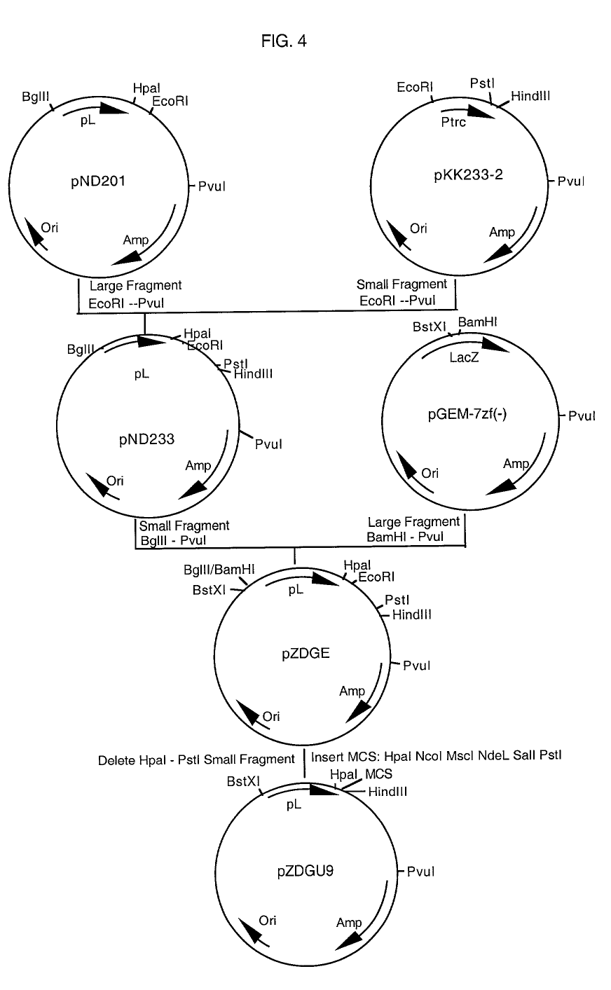

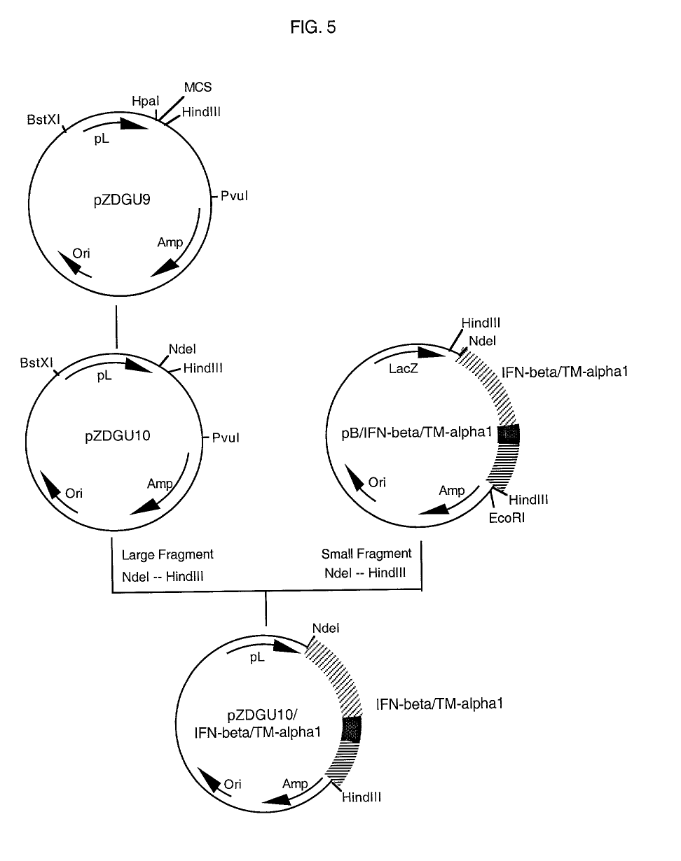

For expression of the IFN-beta/TM-alpha1 fusion gene, the plasmid pB/IFN-beta/TM-alpha1 was digested with restriction endonucleases NdeI and HindIII to release the DNA insert encoding IFN-beta/TM-alpha1. The DNA fragments were gel purified and then ligated to the prokaryotic P_{L} (phage lamda left promoter) expression vectors pZDGU10 (FIG. 5) through the NdeI and HindIII sites. After ligation, the DNA was transformed into competent E. coli strain K12 delta-A delta-trp for expression plasmid-born IFN-beta/TM-alpha fusion proteins. K12 delta-A delta-trp [delta-lacI, M72, rpsL, lacZam, delta(bio-uvrB), delta-trpEA2, limbda, Nam7, Nam53, cI857, delta-H1 (cro-F-A-J-b2), F.sup.minus] (ATCC# 35952) contains the temperature-sensitive cI857 mutation. P_{L} expression vector pZDGU10 was derived from pZDGU9 according to FIG. 4. The plasmid isolated from one of the colonies was confirmed by the analyses of restriction endonucleases and DNA sequence in both directions to comprise a DNA sequence (SEQ ID NO:10) encoding a human IFN-beta/TM-alpha1 fusion protein (SEQ ID NO: 11). The plasmid is designated as pZDGU10/IFN-beta/TM-alpha1 (FIG. 5).

K12 delta-A delta-trp cells were used for expressing plasmid-born IFN-beta/TM-alpha fusion proteins. 35 microliter of K12 delta-A delta-trp competent cells were thawed on ice and transferred into an eppendorf tube containing approximately 5 ng pZDGU10/IFN-beta/TM-alpha1 DNA. The mixture is left on ice for 30 minutes and mixed by swirling gently. The cells are heat-shocked at 42.degree. C. for exactly 45 seconds in a circulating water bath that has been preheated at 42.degree. C. The cells are rapidly returned to an ice bath and allowed to chill for 10 minutes. Ten volumes of SOC medium are added to the tube. The cells are incubated at 28.degree. C. for 60 minutes with shaking at 250 rpm to allow the bacteria to recover and to express the antibiotic resistant marker encoded by the plasmid. Transformed competent cells are transferred onto 90-mm agar plates containing the antibiotic and gently spread over the surface of the agar plate using a sterile bent glass rod. The plates are left at room temperature until the liquid has been absorbed. The plates are then inverted and incubated at 28.degree C. overnight.

Plasmid pZDGU10/IFN-beta/TM-alpha1 is deposited with the American Type Culture Collection (ATCC) as a patent deposit at 10801 University Blvd., Manassas, Va. 20110: Accession number: PTA-2867; Deposit date Dec. 20, 2000 (E. coli K12 delta-A delta-trp/pZDGU10/IFN-beta/TM-alpha1 as the host vector system). Plasmid pZDGU10/IFN-beta/TM-alpha1 is a recombinant expression vector comprising a DNA sequence (SEQ ID NO: 10) encoding a human IFN-beta/TM-alpha1 fusion protein. The fusion protein comprises human IFN-beta (amino acids 1-166) linked to human TM-alpha1 (amino acids 172-199) via a peptide linker (amino acids 167-171), as shown in SEQ ID NO: 11.

The E. coli K12 delta-A delta-trp cells containing the recombinant expression vector pZDGU10/IFN-beta/TM-alpha1 were grown overnight in Luria Broth (LB) containing 100.mu.g/ml ampicillin at 28.degree. C., with rotary shaking at 225 rpm. The overnight culture was diluted 1:50 into a fresh LB medium with 50 mu.g/ml ampicillin. The E. coli K12 delta-A delta-trp cells were grown at 28.degree. C. until the OD.sub.680 of the culture reached 2.0. The expression of plasmid-born IFN-beta/TM-alpha1 was induced by raising the temperature to 42.degree. C. At this stage, zinc sulfate (ZnSO₄) was added to give a final concentration of 0.5 mM ZnSO₄ for stabilization of IFN-beta/TM-alpha1 synthesized in E. coli (Gross et al., Biochi. Biophy. Acta., 825:207, 1985). The cultivation is continued for another 5 hours in that matter. The cells were harvested by centrifugation and the bacterial pellets were stored at −80.degree. C. until further purification.

For purification of IFN-beta/TM-alpha1 fusion proteins, the frozen E. coli cell pellets were suspended in 6 volumes of ice-cold lysis buffer (50 mM Tris HCl, pH 8.0, 1 mM EDTA, 1 mM DTT, 1 mM phenylmethanesulfonyl fluoride, 2 mg/ml lysozyme) and disrupted by sonication and two cycles of quick freezing/thawing in liquid nitrogen and 37.degree. C. water bath. The cell lysate was centrifuged at 4.degree. C. and the supernatant was diluted with an equal volume of ice-cold lysis buffer. Saturated ammonium sulfate was added by dropwise into the supernatant with constant mixing to a final concertation of 33% ammonium sulfate saturation. After 20 minutes on ice, the precipitate was collected by centrifugation at 4.degree. C. and resuspended in ice-cold 20 mM sodium phosphate buffer (pH 7.0) containing 300 mM NaCl. The supernatant was further purified to homogeneity in a sequential immunoaffinity (Daniela et al., J. Gen. Virol., 64:905, 1983) and metal-affinity (Edy et al., J. Biol. Chem., 252:5934, 1977) column chromatographic purifications.

Briefly, the crude extract was loaded onto an immunoaffinity column equilibrated with 20 mM sodium phosphate buffer (pH 7.0) containing 300 mM NaCl. The affinity column was washed with the equilibrated buffer until the absorbance of the eluate is zero or nearly zero, and then eluted with acetate buffer (100 mM acetic acid, pH 2.0, 300 mM NaCl). The fractions having antiviral activity were pooled and then adjusted to 50 mM sodium acetate buffer (pH 5.0) and applied to a zinc chelating affinity sepherose column. After washing 50 mM sodium acetate buffer (pH 5.0), IFN-beta/TM-alpha1 was eluted with 100 mM sodium acetate buffer (pH 4.0) containing 150 mM NaCl and 150 mM imidazole. All the purification steps were carried out at 4.degree. C.

The IFN-beta/TM-alpha1 fusion protein examples were analyzed under standard reducing conditions in 15% SDS polyacrylamide gel electrophoresis (SDS-PAGE) (Laemmli, Nature, 277:680,1970). The protein bands are visualized by Coomassie blue staining. The apparent molecular weight of the fusion protein is 23 kD in agreement to the calculation from the amino acid composition. When examined by Western blot (Towbin et al., Proc. Natl. Acad Sci (USA) 76:4350, 1979; Burnette, Anal. Biochem. 112:195, 1981), it is found that the IFN-beta/TM-alpha1 contains human IFN-beta component. The concentration of the fusion proteins was determined with the BioRad Protein Assay. This assay uses the dye Coomassie brilliant blue and measures the protein/dye complex at 595 nm. The standard used is bovine serum albumin.

### EXAMPLE 3

Antiviral Properties of IFN-beta/TM-alpha1 Fusion Proteins

One of the biological assays for the fusion protein comprising IFN-beta and TM-alpha1 was an antiviral assay. Antiviral specific activity of the fusion protein was determined on human cells by using cytopathic effect (CPE) inhibition assays as reviewed previously (Stewart, The Interferon System, Springer-Verlag, 17-18, 1979; Rubinstein et al., J. Virol. 37:755, 1981; Famillett et al., Meth. Enzymol. 78:387,1981). Briefly, 100 .mu.l of WISH (human amniotic cell line, ATCC) cells suspension (4 times10.sup.5 cells/ml) were seeded in 96-well microplates, respectively. 100.mu.l of two-fold serial diluted interferon preparations was added to each well. After incubation for 24 hours at 37.degree. C. and 5% CO.sub.2 in air, the cells were infected with vesicular stomatitis virus (VSV) (Indiana strain, ATCC), followed by an additional 24 hours incubation. Every sample was done in triplicate. The CPE was checked under a microscopy on virus control, cell control and cells which received NIH standard interferon. The highest dilution giving 50% reduction of the viral plaques was considered as the end point. The interferon unit was defined as the reciprocal of the dilution at the 50% endpoint and was adjusted to the NIH interferon reference standard (Gb23-902-531). The results are reported in Table 7 below.

TABLE 7
Antiviral activity of fusion proteins using VSV as the challenge virus

| | Specific activity/mg protein |
|---|---|
| Interferon | WISH |
| IFN-beta | 8.0.times.10.sup.7 lu |
| IFN-beta/TM-alpha1 | 6.2.times.10.sup.7 lu |

The specific biological activity of the IFN-beta or the IFN-beta/TM-alpha is presented as the number of biological units per mg of the total protein present. The data in Table 7 show IFN-beta/TM-alpha1 has a similar titer as IFN-beta in the CPE inhibition assay on WISH cells.

### EXAMPLE 4

Immunological Activity of IFN-beta/TM-alpha1 in E-Rosette Assay

The E-rosette bioassay performed in this invention is based on the observations that the addition of optimally active thymosin preparation can increase in patients with thymus hypoplasia the percent and absolute number of peripheral blood T cells forming rosette with sheep red blood cells (Wara et al., N. Engl. J Med. 292:70, 1975), and that thymic extracts can restore the erythrocyte rosette-forming capacity of alpha-amanitin-treated lymphocytes (Sattar et al., Immunol. Lett. 27:221, 1991). In fact, the percentage of E-rosette forming cells in peripheral human blood can be a measure of the content of fully mature T-cells. In a healthy adult the normal level of E-rosettes is about 56%. For the performance of the E-rosette assay, a RNA polymerase inhibitor, alpha-amanitin, was used. In brief, human peripheral blood lymphocytes were separated by Ficoll-Hypaque gradient centrifugation, washed and resuspended in RPMI. After being blocked with alpha-amanitin, the cells were incubated with varying concentrations of either synthetic TM-alpha1 or the IFN-beta/TM-alpha1 fusion protein, followed by addition of sheep red blood cells. A rosette was defined as a lymphocyte that bound three or more sheep erythrocytes. Rosettes enumerated under a microscope by counting 200 lymphocytes. The results were expressed as percent lymphocytes forming rosettes. The value of the normal level of E-rosette in the healthy adult is taken as 100% in a relative numerical scale, and after the alpha-amanitin blockage it is taken as 0%. Each data point was done in duplicate. The results are shown in Table 8.

TABLE 8
E-rosette assay in comparison of the fusion protein with synthetic TM-alpha1

| Synthetic TM-alpha1 | E-rosette number | IFN-beta/TM-alpha1 | E-rosette number |
|---|---|---|---|
| (.mu.g/0.5 ml culture) | (%) | (.mu.g/0.5 ml culture) | (%) |
| 6.0 | 30 | 5.0 | 19 |
| 3.0 | 52 | 2.5 | 43 |
| 1.5 | 62 | 1.25 | 57 |
| 0.75 | 100 | 0.63 | 91 |
| 0.38 | 57 | 0.31 | 51 |
| 0.19 | 34 | 0.156 | 21 |

In a comparison of synthetic TM-alpha1 with the fusion protein, it appears in the E-rosette assay that the IFN-beta/TM-alpha1 fusion protein shows a similar immunological action to synthetic TM-alpha1 and possess the TM-alpha1's action on the differentiating mechanism and on the maturation of thymus-related lymphocytes to immune-competent T cells.

I claim:

1. A DNA sequence encoding a fusion protein comprising IFN-beta and TM-alpha1, as shown in SEQ ID NO:10.

2. A DNA sequence encoding a fusion protein according to claim 1, wherein IFN-beta is fused to TM-alpha1.

3. A DNA sequence encoding a fusion protein according to claim 2, wherein IFN-beta is fused to TM-alpha1 via a peptide linker.

4. A DNA sequence encoding a fusion protein according to claim 3, wherein the fusion protein comprises IFN-beta/Ser(Gly)₄/TM-alpha1.

5. A DNA sequence encoding a fusion protein according to claim 4, wherein the fusion protein consists of the amino acid sequence of human IFN-beta (amino acids 1-166), human TM-alpha1 (amino acids 172-199), and a peptide linker (amino acids 167-171), as shown in SEQ ID NO:11.

6. A recombinant expression vector comprising a DNA sequence according to claim 1.

7. A plasmid according to claim 6, where the plasmid is designated as pZDGU10/IFN-beta1/TM-alpha1 deposited with the American Type Culture Collection under accession number PTA-2867.

8. A host cell containing a vector according to claim 6, wherein said host cell is a mammalian, plant, insect, yeast, or bacterial cell.

9. A process of preparing a fusion protein comprising IFN-beta/TM-alpha, including the step of culturing a suitable host cell containing a vector according to claims 6 under conditions promoting expression.

10. A composition comprising a pharmaceutically effective amount of a fusion protein as claimed in claim 9, and a pharmaceutically acceptable carrier.
